(19) United States
(12) Patent Application Publication
(10) Pub. No.: US 2005/0107825 A1
(43) Pub. Date: May 19, 2005

(54) Surgical method for mandibular angle fracture operation and devices therefor

(76) Inventor: Hee-Young Lee, Kunsan (KR)

Correspondence Address: DILWORTH & BARRESE, LLP, 333 EARLE OVINGTON BLVD., UNIONDALE, NY, 11553, US

(21) Appl. No.: 11/019,988

(22) Filed: Dec. 21, 2004

(30) Foreign Application Priority Data
Dec. 24, 1999 (KR) 62165-1999
Jan. 27, 2000 (KR) 3904-2000
Feb. 1, 2000 (KR) 4946-2000

(51) Int. Cl. A61B 17/50

(52) U.S. Cl. 606/210

(57) ABSTRACT

A novel type of method for mandibular angle fracture operation requiring only incision of lateral of mandible and its devices are provided. Only 20 mm of the incision of mandible makes it possible for an operator to perform the operation and to save time and recover time without incision of patient's face because the operator uses the patient's mouth to broaden the operator's view. A mandibular retractor comprising a handle, a frame, an activated shaft having a tip and a controller lever is provided for cutting or holding the target area of the mandibular. A drill-driver comprising a handle, a neck, a power transferring member, a head including a tip hole and an operation axis and a power connection member provided for fix a metal plate on the bone. A tweezers comprising a body portion, a pair of leg portions which are twisted by 90 degree about the body portion and paralleled each other is provide for holding a bolt or nut.

### BACKGROUND OF THE INVENTION

1. Field of the Invention

The present invention relates to a surgical method for mandibular angle fracture operation and its devices and, more particularly, a mandibular angle fracture operating method for mandibular angle fracture surgery without incising a patient's face, and its surgical instruments including a mandibular retractor, a drill-driver, and a pair of tweezers.

2. Background Art

In plastic surgeries and mandibular angle fracture surgery procedures, it has usually been required to incise a face portion of a target area. This method causes many difficulties for operators and assistants. For example, a scope of the operator's sight for the operation is limited and the operation under a limited view for a target area needs longer operating hours. In addition, these difficulties require efficiencies and surgical skills of operators and assistants. Scars can remain on a patent's face and it can take a long time to recover. Especially, the operation of mandibular angle fracture is performed in a limited area of a patient's mouth. Therefore, there are needs to use a surgical method and its instruments that are easily used in a narrow area of a patient's mouth.

Surgical instruments including a mandibular retractor, a drill-driver, and a pair of tweezers are provided in the present invention.

A conventional mandibular retractor (FIG. 10) consists of a handle and a loop on a tip of a retractor and is bent by 45 degrees at a neck area. This structural shape is designed for a loop retracting a mandible so that an operator has a certain degree of field of vision. However, using the existing mandibular retractor only functions for providing a field of vision and it cannot be used for fixing metal plates to the bone or cutting target bones that are always needed for a procedure of the surgery as shown in FIG. 11. In surgical situations to fix metal plates on mandibular inferior border for mandibular angle fracture operation, using the mandibular retractor interfere with an operator's view and cannot hold bone particles so that it requires assistants holding them with his fingers. In mandibular angle resection, the bone particles being produced from resection often remain behind the mandible and an operator wastes time to find and remove them. Another trouble is that it does not include a lighting device, so a separate lighting has to be attached to light the target area. Using separate light decreases the operator's field of view and an electric wire interferes with the operator's surgical performance. Relating to a conventional drill and a conventional screwdriver, they are separately used to fix metal plates on the surface of bone. The conventional drill and screwdriver have 15 mm thickness head portions and a drill tip or screw tip has 25 mm length. Therefore, the 20 mm incision required in the present invention will not be enough for operation when the conventional devices are used. In this case, an additional incision on the face of patient for inserting the drill and the screwdriver (2-j) is needed. This causes the following problems: longer operating time, lack of surgical efficiency by working two separated areas (FIG. 20), and longer time for patient's recovery. And fixing metal plates on the bone is not completely ensured because of inserting the drill and the screwdriver in a slant line. There is also an economical problem that is caused by purchasing two different instruments: a drill needs fast revolution while a driver needs slow speed for each surgical occasion.

A pair of tweezers are used in mandibular angle fracture operation for holding or fixing metal plates on the broken area. The tweezers place and temporarily hold metal plates on a target area until drilling holes are made for bolts. Existing tweezers are not exclusively designed for mandibular angle fracture operation, so the operation with them requires more skill and experience. Difficulties with using the tweezers include the following: the tweezers cannot hold a metal plate and bolts simultaneously, and cannot be easily handled when they are used with a screwdriver, and the tweezers need human power to squeeze them as the operation takes longer time more and additional help will be needed.

Therefore, the present invention discloses a new mandibular angle fracture operating method without facial incision and surgical instruments including a mandibular retractor, a drill-driver and a pair of tweezers to overcome the difficulties of the prior art.

### SUMMARY OF INVENTION

The present invention is a novel method for mandibular angle fracture operations in that only a lateral incision at the mandible is required without incision of face. Even though an operator gets about 20 mm of operation space from the incision of the mandible, the operator's field of vision will be broadened as much as the size of patient's mouth and the operation procedure will be easier while working in the mouth. In addition, surgical instruments including a mandibular retractor, a drill-driver, and a pair of tweezers for the mandibular fracture operation method are provided. The mandibular retractor comprises a handle, a frame which defines a hook shaped loop at the end thereof and has inner hollow groove and at least an actuation member including an activated shaft having a tip and a controller lever. The drill-driver comprises a handle, a neck, a power transferring member installed in the inner hollow space of the neck, a head including a tip hole and an operation axis and a power connection member including bevel gears. The tweezers comprises a body portion, a pair of leg portions which are twisted by 90 degree about the body portion and paralleled each other. The end of the leg portions defines mouth portions with a circled groove for bolts and a side groove for a metal plate.

### DETAILED DESCRIPTION OF THE PREFERRED EMBODIMENTS

The present invention provides a surgical method of mandibular angle fracture operation. The method includes a step of incising a lateral portion of mandibular in a patient's mounth and maintaining a 20 mm distance space from the bone surface for inserting surgical instruments including one or more of a mandibular retractor, a drill-driver, and a pair of tweezers. The operation can be performed without incision of face because an operator can get a good field of vision through the 20 mm space from incising the lateral portion of mandibular and a patient' mouth.

Referring to FIGS. 1-10, a mandibular retractor consists of a handle (1), a frame (2) which is bent 45 degree from the handle (1) and an actuation member including an activated shaft (3) having a tip (3a) and a control lever (4). The frame (2) includes a hook-shaped loop (2a) at the end thereof, and an inner hollow groove space (2b) for installation of the activated shaft (3) having an tip (3a) and a control lever (4) which is squeezed to operate the activated shaft (3) having the tip (3a). FIG. 1 is a general structure of the mandibular retractor and FIG. 2 is showing how each part including a handle (1), a frame (2), and a lever (4) is working together.

The cut end of the frame (2) is rectangular in shape. The connecting part between the handle (1) and the frame (2) includes ‘flat part (2c)’, and bent part which is called ‘bent part (2d)’. The end of the frame (2) defines a semi-circular shaped loop (2a).

The frame (2) includes an inner hollow groove (2b) for installation of the activated shaft (3) and a groove cover (5). The groove cover (5) is removable to replace various activated shafts for the activated shaft (3) for various characteristics of an operation. There is a tunnel (2f) through which the lever (4) is inserted and which is connected with the frame (2) via an axis (4a) for pivotable movement of the lever (4) to operate the activated shaft (3).

The lever (4) is a power source for the end tip (3a) of activated shaft (3). The loop (2a) and the tip (3a) work together for fixing, holding or cutting by grasping power of the lever (4). Grasping power is carried by the head (4b) and the handle (4c) of the lever (4).

The head (4b) of the lever (4) and the activated shaft (3) either can be connected directly to each other or connected via a pushing shaft (6). On both end of the pushing shaft (6), there are holes (6a, 6b) for assembling other parts including the lever (4) and the activated shaft having the tip (3a).

The activated shaft (3) has a flexible portion made of a wire (3d) with rods (3c), and a plate spring or a chain for flexibility as shown in FIGS. 5a-b. The wire (3d) with rods (3c) delivers grasping power to the tip (3a) to be moved forward and backward. And it can be applied in a twisted direction to provide various movements and for operation in hard-to-reach locations as shown in FIG. 3. The tips (3a) are also used for various purposes by switching tips for each purpose as shown in FIGS. 6a-c. FIGS. 7-9 shows examples for fixing, holding or cutting the bone.

There is a clamp (7) for maintaining a certain position of the actuation member. One side of clamp (7) includes teeth for holding and a lock (7b) for controlling for the movement of lever (4). Therefore, both the teeth and the lock (7b) allow an operator to perform without assistants' help holding a metal plate, pressing on it or removing cut bone particles.

Also, this invention provides a lighting (9) for securing field of view for operation. The lighting device consists of a battery case (9a) for a power source, a light bulb (9b) located on a bent part (2d), and a mirror (10) installed on the frame (2) and the frame part functions as a mirror. The lighting device which is attached to the main body is designed for easier operation and the mirror (10) reflects light for measuring distance to a target area and securing view range.

So this new mandibular retractor not only maintains its common function as a retractor but also performs additional roles such as cutting, holding and fixing the bone and lighting features.

Referring to FIGS. 11-18, a drill-driver has a handle (120) consisting of a front handle (121) and a rear handle (122), a neck (130) having a inner hollow groove space, a head (140) including a tip hole (151) and an operation axis (150), a power connection member (160) installed in the front handle, a manual selecting lever (180), and a power transferring member installed in the inner hollow groove space of the neck (130). A power source member (110) is provided in the rear handle (122). The power source member (110) consists of a motor and a reducing speed gear to reduce rotating speed (111). A reducing speed motor for reducing rotating speed may be provided instead of a motor and a reducing speed gear. A reversing motor may be provided. A reversing switch (112) or a reversing gear may further be provided. The power source member (111) may be a high speed power source (a) or low speed power source (b) which may be installed in the rear handle (122). A user can replace the rear handle (122) for another rear handle to choose the high speed power source (a) or the low speed power source (b). (FIG. 13 shows a structural description for the handle parts.) The high speed source (a) is used for drilling while the low speed source (b) is used for screwing. The high speed source (a) or the low speed source (b) can be decided according to the ratio of the reducing speed by the reducing spped gear. The rear handle (122) including the power source member is easily hooked up by clicking parts to the front handle (121). In the high speed power source (a), the shaft (111a) of the motor is connected to a first shaft (161) of power connection member installed in the front handle (121) as shown in FIG. 14. The low speed power source (b) further includes a torque controller (170), which keeps a certain level of twisting pressure for screwing. The torque controller (170) acts like an electric fuse to control and deliver adequate power to twist for preventing bone destruction because of high twisting pressure. As shown FIGS. 15a-b, the torque controller (170) consists of a torque controller shaft connected to the first shaft (16) of power connection member, a sliding shaft (172) installed in the shaft (111a) of motor, and round disks (171a, 172a) between them. When the pressure of twisting power is more than a certain level, the power from the power source is not transferred to device any more to prevent bone destruction because the disks (171a, 172a) slip each other and do not turn together.

The neck (130) is a cover of power transferring member and provides protection for the parts and is extended form the head. The neck (130) is shaped like cylinder or square and the power transferring member is installed therein.

The head (140) is bent with 15-25 degrees at the neck and includes an operation axis (150) and a tip hole (151). The head (140) is designed for working and maintaining perpendicular to the bone to make the operation easier regardless of the shape of the bone for drilling and crewing bolts. The thickness of the head (140) is 6-7 mm so as to have an enough space to operate when the head (140) is put in the mouth. Because 20 mm incision is generally performed, the operator needs to drill as deep as 8-9 mm and the length of drilling tip for drilling is about 10 mm. Therefore, the thickness of head should not be more than 6-7 mm. The operation axis (150) delivers twisting power from the motor to a drill tip (2-a) or a screwdriver (2-b). Either the drill tip (2-a) and the screwdriver (2-b) is fit into the tip hole (151). The transferring member transfers the twisting power from the power source to the head (140) via the power connection member. The power connection member includes a first gear with a first shaft (161) and a second gear (162a) with a second gear shaft. The gears are bevel gears perpendicularly combined with each other to transfer the twisting power.

FIG. 16a shows one configuration of power transferring member for delivering power. A belt (163) is provided for transferring the power. The belt (163) is connected with a first belt pulley installed on a second gear of the shaft of the power connection (161), and a second belt pulley (162b) of the operation axis (150). A bearing member (164) is installed in the neck to prevent power loss when the belt is bent according to the shape of the neck (130).

FIG. 16b shows another configuration of power transferring member for delivering power. The configuration includes a connecting axis (165) having two bevel gears at the end thereof. The connecting axis (165) is connected with the second gear (162a) of the power connection member and the operation axis (150). The both ends of the connecting axis (165) define bevel gears, an upper gear and a lower gear. The upper gear is connected to the second gear while the lower gear is connected to head gears.

Head gears including a bevel gear and normal gears are designed for delivering power from the connecting axis (165) to the operation axis (150). FIG. 17 shows how the gears work together.

A manual selecting lever (180) is provided to operate the device manually and consists of a lever handle (181) that is designed to control pressure twisting bolts and a lever shaft (184). The lever shaft (184) is connected to the second gear (162a) of the power connection member. The lever (180) performs a function like a switch for the power source.

FIG. 18a is showing how the manual selecting lever works. When the lever handle (181) is turned, an end blade (162a) is rotating together. The turning power of the level is transferred to the second gear of the power connection member to rotate the operation axis. The lever is easily detachable from the power connection member. When it is rotated by power it should be detached from the second gear (180) of the power connection member. FIG. 18b shows how to attach/detach the wheel shaped lever to the handle. The wheel shaped lever is located in the front handle (121) to get supported by spring (184), and then various shapes of grooves (182) on the second gear is assembled to the shape of lever shaft (183).

Referring to FIG. 19, the drill-driver is used to fix bolts. Lateral (2-d) of mandibular angle (2-c) is incised and an incision part (2-e) is formed to maintain (2-f) the space and the head of drill-driver is inserted through patient's mouth and fractured area (2-g) is drilled and is covered by the metal plate (2-h). The room from the incision of lateral of mandibular is approximately 20 mm, and thickness of the head of the drill-driver is limited to 7 mm. Therefore, the length of all instruments should be less than 17 mm to operate freely. In addition to the length, the head of the drill-driver is twisted by 15-25 degrees to easily approach to the bolts at a right angle.

The drill-driver described above may be used for the new operation and thereby saving operation time and recovery time.

Referring to FIGS. 21-26, a pair of tweezers are described. The tweezers are made of thin flexible metal and consist of a body portion (201), and a pair of leg portion (202). The end of the portions defines mouth portions (203) with circle grooves for bolts and side grooves for a metal plate (3-a). The body portion (201) is designed for providing flexibility and whole parts of the body are made of same metal. The leg portions (202) are twisted by 90 degrees bout the body portion (201) and parallel each other. The circle grooves for bolts and the side grooves for a metal plate (3-a) are shown in FIGS. 21-24. The grooves for a head of a bolt and a metal plate are designed for easily placing them and maintaining operator's view. As shown in FIG. 24, each leg's width is less than 3 mm, thickness of legs is less than 2 mm, and the height of groove for metal plate is 1 mm. As shown in FIG. 23a, even though bolts are inserted in the metal plate, bolts do not bother the tweezers' movements. When bolts are applied on the tip of the drill-driver, it does not require pre installation of bolts on the metal plate while the metal plate is being held by the tweezers. Stoppers (206) are provided with the mouth portion (203) to fix the metal plate to the groves.

As shown in FIG. 25, the metal plate (3-a) is inserted deeply in the side grooves of the mouth portion (203) to be held by the stoppers (206). As shown in FIG. 23a, when bolts are located on the metal plate (3-b), the metal plate (3-a) is fixed by the circle grooves. The tweezers further includes an outer clamp (207) which are provided to keep a certain position of the tweezers without grasping power. Outer clamp (207) consists of a screw axis (207a) and a pinch nut (207b). The screw axis (207a) goes through the body portion of the tweezers and the nut (207b) combines and adjusts the distance of the leg portions of the tweezers. With this feature, an operator does not have to press the body portions of the tweezers with his fingers during the operation. A certain distance gap between the legs (202) can be maintained without grasping the tweezers. The mouth portions (203) of the legs (202) are designed to be bent to maintain a certain degree of angle between target operation areas and the approaching direction of the tweezers to easily place the metal plate on fractured bone as shown in FIG. 26. The bent degree of leg is preferably 20 degree considering mandibular angle (3-e).

As shown in FIG. 27, the metal plate (3-a) and bolts (3-d) are held together by the tweezers which are placed on the fractured bone (3-f). Fixing the metal plate and twisting a bolt with the drill-screwdriver (3-g) half way, then the metal plate is not moving around on the surface of bone. Next step will be releasing the tweezers and using tips on the tweezers to adjust metal plate on a right position and fixing bolts.

The tweezers are designed to twist the leg portion at 90 degree against to the body so that the legs lie parallel to surface of bone. And this structure provides more view field and reduces the space that the tweezers are taking up in patient's mouth so that the operator can freely move the surgical instruments and required less effort during the operation. Each leg can be adjustable by rolling bolts and even this function is used to hold metal plate like clamp unlike existing tweezers. This feature provides less stress and more concentration to the operator. The tweezers can be used with the drill-driver and can be used to fix the metal plate with or without bolts.

### BRIEF DESCRIPTION OF THE DRAWINGS

FIG. 1 illustrates a perspective view of a mandibular retractor and an exploded view of frame portion of the mandibular retractor according to the present invention;

FIG. 2 illustrates a perspective view of a mandibular retractor according to the present invention;

FIG. 3 illustrates a side elevational view of another embodiment of mandibular retractor having a twisted frame according to the present inventions FIG. 4 illustrates a sectional view of a mandibular retractor according to the present invention when the mandibular retractor is activated;

FIGS. 5a and 5b illustrate a perspective view of activated shaft having an tip and a partial sectional view of FIG. 5a;

FIGS. 6a-c illustrate perspective views of different types of the tip;

FIGS. 7-9 illustrates a perspective view and sectional views showing the methods of using different types of the tips;

FIG. 10 illustrates a perspective view of a conventional mandibular retractor;

FIG. 11 illustrates a perspective view of a drill-driver according to the present invention showing an internal configuration and a drill tip or a screw;

FIG. 12 illustrates an elevational view of a drill-driver according to the present invention;

FIG. 13 illustrates a partial elevational and partial sectional view of the handle portion of the drill-driver and a sectional view of the rear handle portion of the drill-driver including a reversing switch;

FIG. 14 illustrates a partial elevational and partial sectional view of the drill-driver when the device is in use as a drill;

FIGS. 15a-b illustrates a partial elevational and partial sectional view of the drill-driver and a sectional view of the handle portion of the drill-driver when the device is in use as a screwdriver;

FIGS. 16a-b illustrates a sectional view of the drill-driver having a belt member and a sectional view of the drill-driver having a connecting shaft member showing a power transferring system;

FIG. 17 illustrates a sectional view of a head portion of the drill-driver shown in FIG. 16b;

FIGS. 18a-b illustrate sectional views of a head portion of the drill-driver in manual positions;

FIG. 19 illustrates a environmental sectional view showing patient's mandible and a drill-driver in use;

FIG. 20 illustrates a environmental sectional view showing patient's mandible and a conventional driver in use;

FIG. 21 illustrates a perspective view of a pair of tweezers according to the present invention;

FIG. 22 illustrates a plain view of a pair of tweezers in use shown in FIG. 21;

FIGS. 23a-b illustrate a perspective environmental view of mouth portion of the tweezers grasping a metal plate and bolts and a front sectional views of the bolts fixed by the tweezers and a metal plate;

FIG. 24 illustrates a plain view and a sectional view of mouth portion of the tweezers;

FIG. 25 illustrates a plain view of the mouth portion of the tweezers holding a metal plate;

FIG. 26 illustrates an environmental view of a pair of tweezers having bent mouth portion;

FIG. 27 illustrates an environmental view of a pair of tweezers in use; and

FIG. 28 illustrates an environmental view of a mandibular retractor, a drill-driver and a pair of tweezers in use while a mandibular angle fracture operation.

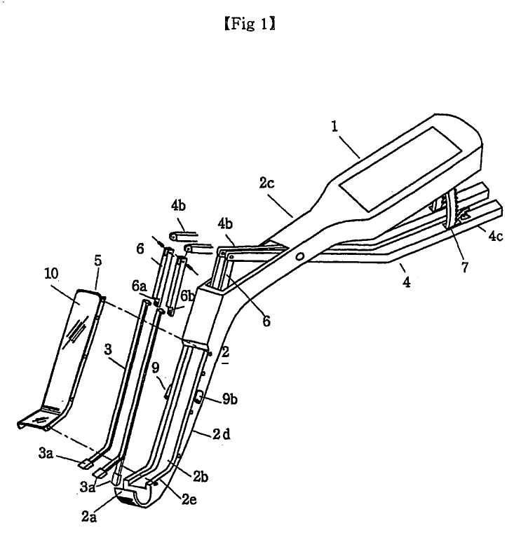

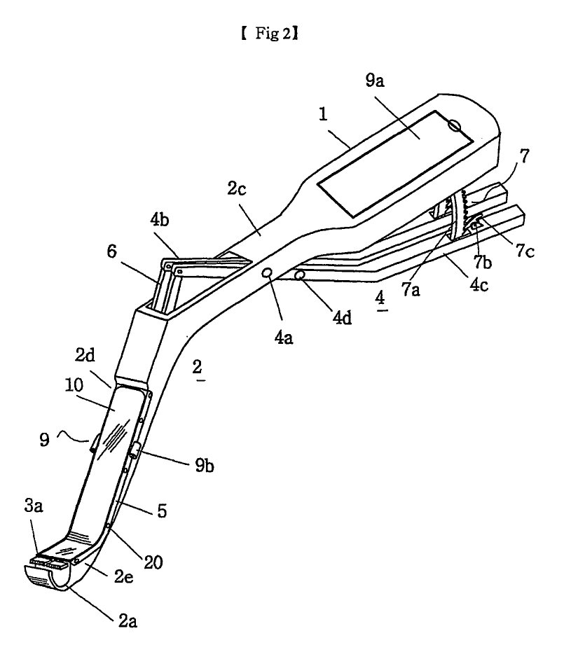

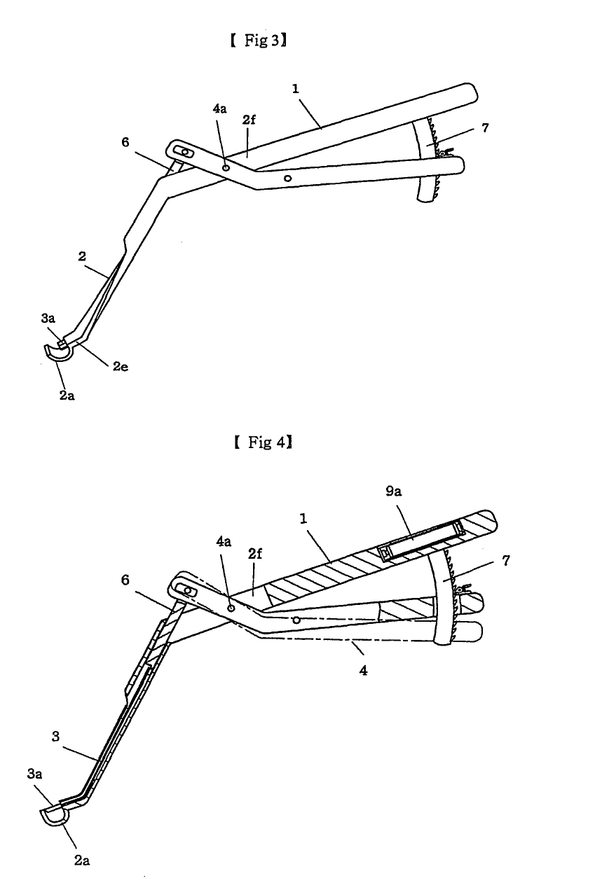

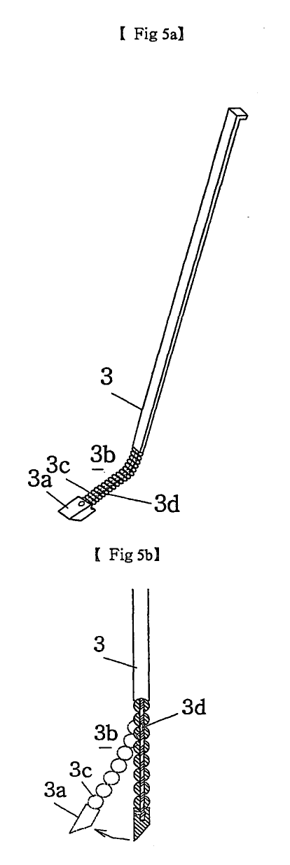

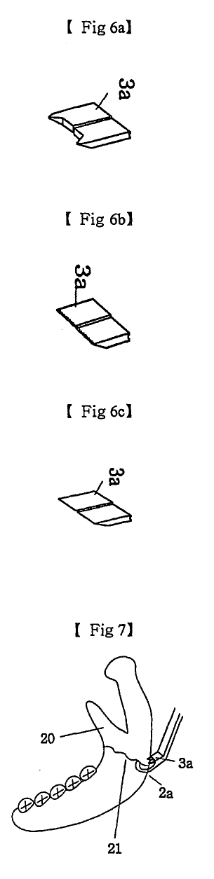

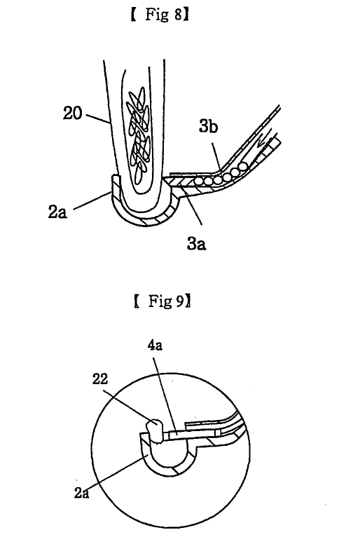

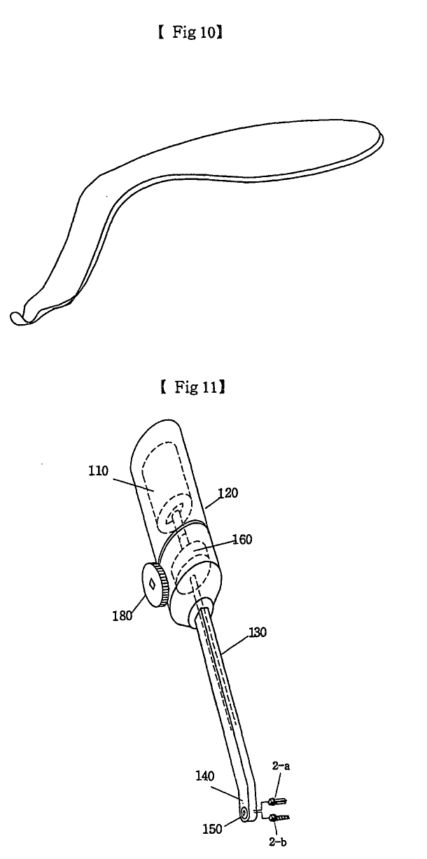

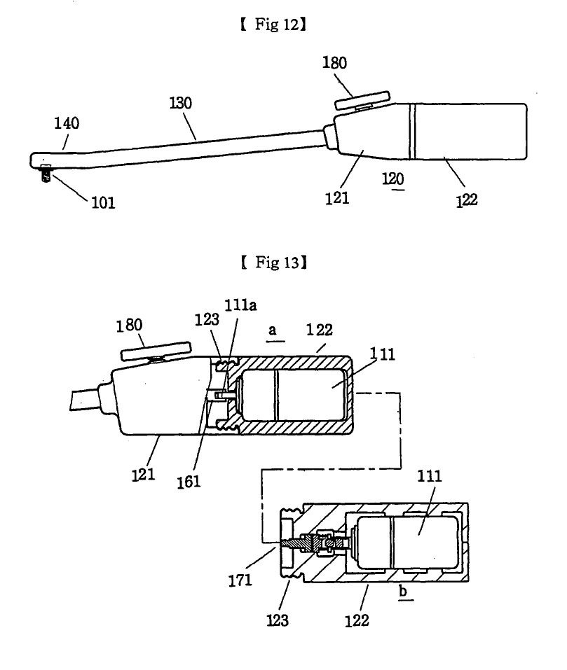

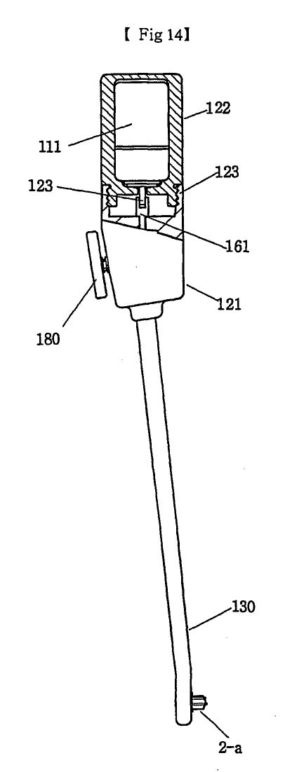

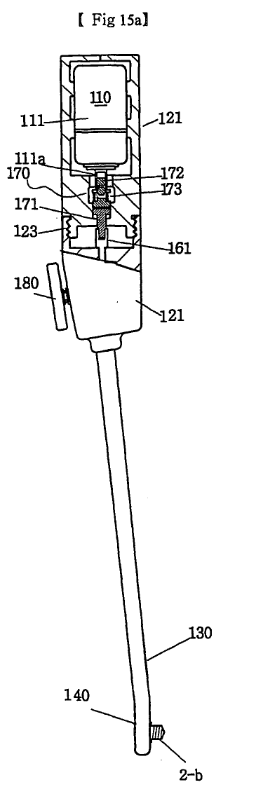

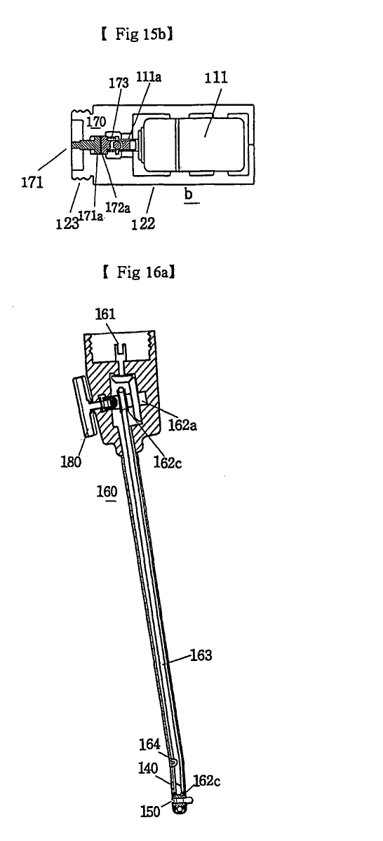

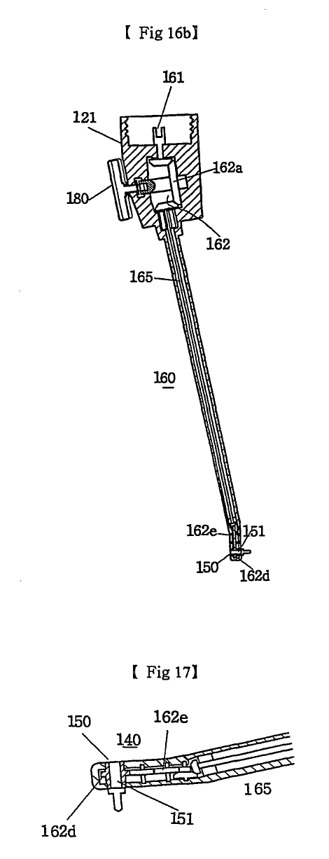

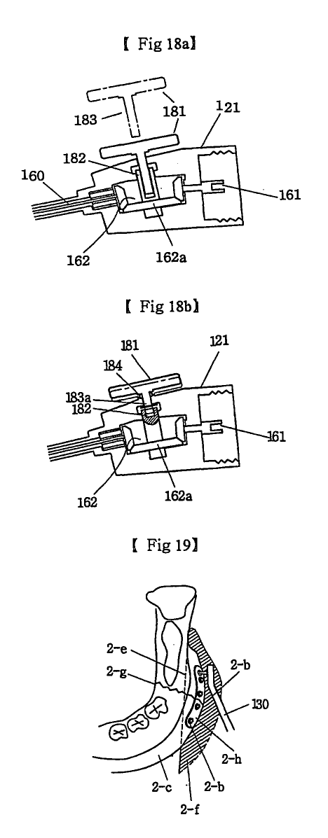

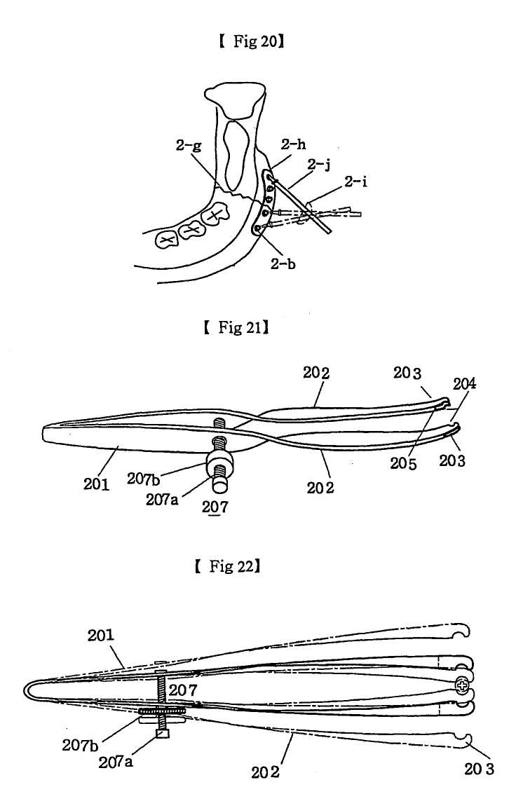

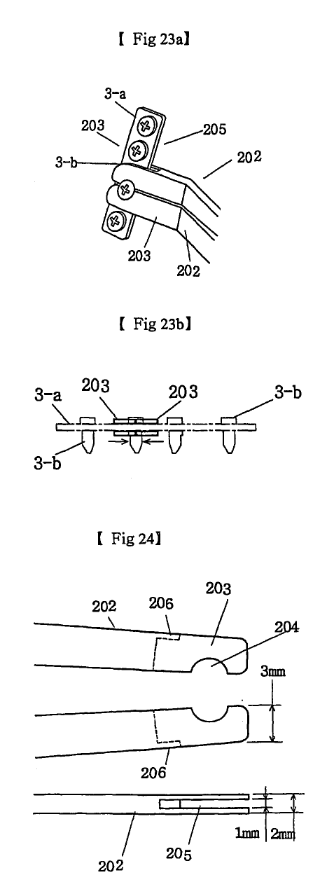

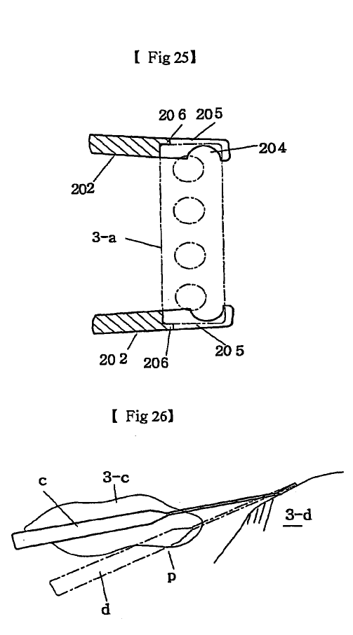

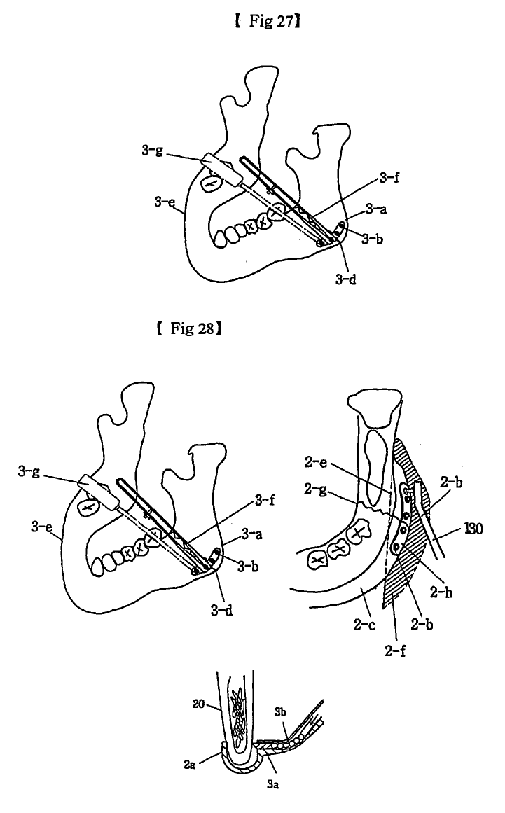

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A pair of tweezers made of thin flexible metal comprising:
a body portion; and
a pair of leg portions which are twisted by a 90 degree angle about the body portion and parallel to each other, the ends of the leg portions defining mouth portions with circle grooves for bolts and a side groove for metal plate.

15. A pair of tweezers according to claim 14, wherein each width of the leg portions is less than 3 mm and each thickness of the leg portions is less than 2 mm, and the height of the side grooves for metal plate is 1 mm.

16. A pair of tweezers according to claim 14, wherein the mouth of the leg portions includes stoppers in the mouth portion of the leg portions.

17. A pair of tweezers according to claim 14, wherein the mouth of the leg portion is bent at a 20 degree angle about the direction of the legs.

18. A pair of tweezers according to claim 14, wherein a clamp is installed to maintain a certain distance gap between the legs without grasping the tweezers.
